(19) United States
(12) Patent Application Publication
(10) Pub. No.: US 2022/0062611 A1
(43) Pub. Date: Mar. 3, 2022

(54) ULTRASOUND TRIGGERED MONODISPERSE SIZE-ISOLATED MICROBUBBLES (SIMBS) TO IMPROVE DRUG DELIVERY

(71) Applicant: Advanced Microbubbles Inc., Newark, CA (US)

(72) Inventors: Jameel Feshitan, Eureka, CA (US); Shashank Sirsi, Dallas, TX (US)

(21) Appl. No.: 17/464,064

(22) Filed: Sep. 1, 2021

Related U.S. Application Data
(60) Provisional application No. 63/073,249, filed on Sep. 1, 2020.
(60) Provisional application No. 63/147,967, filed on Feb. 10, 2021.

(51) Int. Cl. A61M 37/00

(52) U.S. Cl. CPC A61M 37/0092; A61M 2205/3375

(57) ABSTRACT

Use of size-isolated microbubbles (SIMs) for sonoporation using a sonoporation system. The sonoporation system may include a SIM product administered in conjunction with an administered drug. Targeted ultrasound energy using an ultrasound device of the sonoporation system may result in sonoporation in response to cavitation of the SIM product in a targeted area of soft tissue. In turn, targeted delivery of the drug to the targeted area may occur. Results demonstrate improved outcomes using much reduced drug doses that allow for side effects and adverse reactions to be reduced or eliminated when used in conjunction with sonoporation as contemplated herein.

### CROSS REFERENCE TO RELATED APPLICATIONS

This application claims the benefit of priority of U.S. Provisional Patent Application No. 63/073,249 filed on Sep. 1, 2020 entitled “ULTRASOUND TRIGGERED MONODISPERSE SIZE-ISOLATED MICROBUBBLES (SIMBS) TO IMPROVE DRUG DELIVERY” and U.S. Provisional Patent Application No. 63/147, 967 filed on Feb. 10, 2021 entitled “ULTRASOUND TRIGGERED MONODISPERSE SIZE-ISOLATED MICROBUBBLES (SIMBS) TO IMPROVE DRUG DELIVERY,” the disclosures of which are incorporated herein by reference in their entireties.

### BACKGROUND

The use of microbubbles has been proposed in a number of applications including in a variety of biomedical applications. Microbubbles generally describe structures comprising a shell surrounding a gas core and having a size (typically expressed as a diameter) from about 0.5 μm to about 25 μm. The shell of microbubbles may be made of different materials, although one contemplated approach includes microbubbles having a lipid shell surrounding a gas core. Regardless of the microbubble type, the use of microbubbles has been explored for use in conjunction with ultrasound devices for a number of purposes. Specifically, application of energy to microbubbles (e.g., after introduction into the body) may result in excitation of the shell and/or gas core of the microbubble to, for instance, resonate the microbubble. This may result in popping or cavitation of the microbubble at a selected location within the body upon selective application of energy thereto.

Accordingly, microbubbles have been proposed for use as contrast agents in medical imaging, drug delivery, extravascular delivery, noninvasive surgery or other approaches. Specifically, ultrasound devices or the like may provide targeted energy delivery to excite bubbles for specific responses in a targeted area in the body. However, continued development of microbubbles and related treatment systems is required to improve clinical outcomes.

### SUMMARY

The present disclosure generally relates to targeting a drug to a target treatment area. This includes administering to a patient a size-isolated microbubble (SIM) product and administering to a patient a dose of a drug. The targeting also includes introducing ultrasound energy from an ultrasound device to the target treatment area in the presence of the SIMs and the drug. In response, the targeting includes sonoporating the target treatment area in response to the ultrasound energy by cavitating the SIMs of the SIM product to improve delivery of the drug to the target treatment area.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other implementations are also described and recited herein.

### BRIEF DESCRIPTION OF THE DRAWINGS

FIG. 1 depicts an embodiment of a method for microbubble production with control of parameters regarding a diffusion force acting on the microbubbles in the solution undergoing size isolation.

FIG. 2 depicts an embodiment of a system for production of a polydisperse solution of microbubbles.

FIG. 3 depicts an embodiment of a separation column in which a solution comprising a polydisperse solution microbubbles may be disposed during the size isolation process.

FIG. 4 depicts a detailed view of various forces acting on the microbubbles in a separation column during centrifugation for size isolation microbubbles.

FIG. 5 depicts an embodiment of a method for control of the number of diffusion parameters for increased yield during size isolation microbubbles.

FIG. 6 depicts a particular stage during the size isolation process for isolation of microbubbles of a target size.

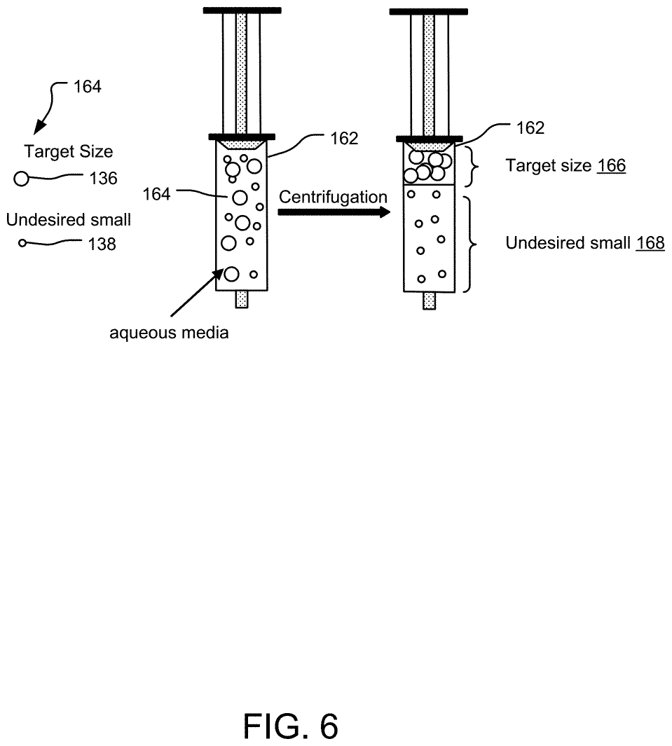

FIG. 7 depicts examples of drug uptake within a tumor showing results from use of a drug in the absence of sonoporation, sonoporation using polydisperse microbubbles, and sonoporation using size isolated microbubbles.

FIG. 8 depicts example results of tumor grown in various different treatment regimens.

FIG. 9 depicts example operations for targeting a therapeutic agent to a target area.

### DETAILED DESCRIPTION

While the presently disclosed technology is susceptible to various modifications and alternative forms, specific embodiments thereof have been shown by way of example in the drawings and are herein described in detail. It should be understood, however, that it is not intended to limit the presently disclosed technology to the particular form disclosed, but rather, the presently disclosed technology is to cover all modifications, equivalents, and alternatives falling within the scope of the presently disclosed technology as defined by the claims.

As noted above, microbubbles have been proposed for use in a number of applications including as imaging contrast agents and the like. However, the present disclosure specifically contemplates use of microbubbles for sonoporation in connection with drug delivery. As will be shown in greater detail below, it has been found that administration of size isolated microbubbles (SIMs) administered with a therapeutic agent allows for use of an ultrasound device for sonoporation of soft tissue. Specifically, SIMs react to targeted ultrasound energy causing the SIMs to cavitate in a region where the targeted ultrasound energy is applied. The cavitation of the SIMS results in sonoporation of soft tissue, which allows for targeted delivery of a drug to vascularized soft tissue, such as a tumor. As will be described in greater detail below, use of ultrasound driven sonoporation of soft tissue tumors using SIMs in combination with therapeutic agents has been shown in numerous animal model and early-stage human trials to slow or halt tumor growth more effectively than therapeutic agents alone.

Much of the proposed work to date using microbubbles has been performed using ultrasound image enhancement agents, which have not been manufactured or designed for drug delivery applications. These agents include microbubbles having a polydisperse size distribution of microbubbles (referred to herein as polydisperse microbubbles or “PMBs”) in sizes ranging from 0.5-20 μm in diameter. Because the size of a microbubble, in large part, determines the physical response in an ultrasound field of applied ultrasound energy, and therefor mechanical effects on tissue, use of PMBs leads to a broad range of unintended tissue effects. Ultrasound image enhancement agents comprising PMBs also have a very short circulatory half-life. The short circulatory half-life of traditional PMBs can limit the time available to deliver therapeutic ultrasound, although, continuous infusion pumps have been suggested to mitigate this issue.

For example, microbubble contrast agents, such as Definity® (available from Lantheus Medical Imaging of N. Billerica, Massachusetts United States), Optison™ (available from GE Healthcare, Chicago, Ill.) or Vevo MicroMarker (available from FUJIFILM Sonosite, Inc., Toronto, Ontario Canada), are polydisperse microbubbles. It has been found that such PMBs have unpredictable behavior when used for ultrasound driven sonoporation. These PMB contrast agents typically contains a range of microbubbles from less than 1 μm to 20 μm diameter. In addition, the microbubbles often have a size below 2 μm in diameter (e.g., a majority of the volume fraction). High polydispersity may be a consequence of the emulsification method used to generate microbubbles in high quantity. Microbubble production methods based on mechanical agitation (e.g., sonication, shaking, and milling) represent the current standard to create encapsulated microbubbles for biomedical applications. Sonication generates large quantities of microbubbles (10¹⁰ per mL) within just a few seconds. However, these methods do not allow homogeneous size distribution of microbubbles, which instead tend to be highly polydisperse due to instability at the water surface causing entrainment of bubbles into the aqueous medium, and subsequent cavitation resulting in bubbles breakup to a critical size (e.g., smaller than 2 μm). Efforts to engineer monodisperse microbubble suspensions have focused on microfluidic technologies including flow focusing, T-junctions, and electro-hydrodynamic atomization. While these techniques may provide low polydispersity, they are rather slow at generating microbubbles and therefore not practical for large-scale manufacturing.

As such, drawbacks in relation to microbubble production and processing have resulted in limitations in the adoption of microbubbles. However, it has been presently recognized that SIMs of a given microbubble target size may be useful in certain contexts. For instance, by controlling the size of microbubbles to a size isolated range, the frequency of the energy applied to SIMS in the body may be adapted for a specific response. Prior proposed methods for microbubble production either result in a polydisperse size distribution or suffer from limitations in microbubble yield such that sufficient quantities may not be feasibly produced for many applications contemplated. In response, certain approaches have been proposed such as those described in U.S. Patent Publication No. 2011/0300078, the entirety of which is incorporated by reference herein.

In addition, PMB agents also exhibit challenges for use in sonoporation of vascularized soft tissues, such as tumors. Sonoporation has shown great potential for drug delivery and gene therapy. Transient plasma membrane perforation achieved by mechanical forces produced from the interaction of focused ultrasound waves with microbubbles increases the permeability of tumor tissues. However, sonoporation of PMBs produces heterogeneous effects leading to complexities and challenges in the realization of controllable and predictable drug delivery. Delivery and monitoring of focused ultrasound energy to tumor tissue has been performed in clinical trials with use of PMB products such as contrast agents that are not optimized for this specific purpose. Effectively monitoring tumor tissue sonoporation in real-time is a critical capability and is important for a successful therapeutic system.

Studies considering the amount of internalized exogenous molecules in sonoporated cells using PMB agents were found to exhibit heterogeneous characteristics. Moreover, sonoporated cells were reported to exhibit heterogeneous and complex concomitant physiological responses, such as calcium-ion transients, calcium oscillations and waves, and non-unitary changes in the levels of plasma membrane potential depolarization. Finally, various cellular developmental effects including proliferation inhibition, cell-cycle arrest, and trends in cell fate (i.e., survival, apoptosis, and necrosis) have been observed in sonoporated cells over several hours following ultrasound exposure. The reason for these heterogeneous effects has yet to be identified. Moreover, for practical applications, the heterogeneity of sonoporation of PMBs poses more challenges in achieving predictable delivery outcome and high delivery efficiency. For instance, some sonicated cells that underwent apoptosis or necrosis are not advantageous to improving the delivery efficiency, because they are essentially the side effects of sonoporation. Also, previous studies have shown that trends in cell fate were correlated with the degree of sonoporation. It is important to address the key acoustic and non-acoustic parameters that are related to the heterogeneity of sonoporation.

The present disclosure recognizes the heterogeneity demonstrated in use of clinically available ultrasound image enhancement PMB products is a major contributing factor to therapeutic effect variability. The need to achieve therapeutic doses of agents within tumors while avoiding the dose dependent side effects requires producing, developing, and deploying a complete system which includes a microbubble agent and hardware. Recognizing this need, the present disclosure includes technology specifically adapted to realize the therapeutic goals of reduced drug dosage requirements in the presence of sonoporation of soft tissue. Specifically, the ability to produce clinically relevant quantities of size iso with requisite qualities of SIMS facilitates the improvements of the sonoporation system presented herein.

Specifically, the sonoporation system contemplated in the present disclosure may provide a SIM solution in connection with a purpose-built ultrasound system to provide for improved sonoporation of vascularized soft tissue tumors. The application of targeted ultrasound energy to vasculature containing the SIM solution causes a sonoporation effect. Specifically, the compressible gas core of the size isolated microbubble product reacts to the oscillating pressure wave by volumetrically expanding and contracting the tissue to temporary (e.g., for a period of 6-24 hours) permeabilize endothelial tissue. In turn, the present system may facilitate increases the permeability of vascularized soft tissue tumors to therapeutic agents. For example, in some examples, permeability of local capillary beds in soft tissue may be limited to molecules larger than around 400 Da. In this regard, the sonoporation contemplated herein may temporarily increase the tissue permeation of circulatory substances to a size than what occurs in the absence of sonoporation. That is, the permeability of local capillary beds in soft tissue in a targeted area may be increased in response to the introduction of ultrasound energy focused on the intended treatment area to cause reversible perforation of capillary walls in specific treatment areas and monitoring said perforation events using diagnostic ultrasound technologies. The increase in permeability in response to the sonoporation may be temporary so as to avoid negative effects such as bleeding or the like in the targeted treatment area. In some examples, sonoporation may temporarily make the local capillary beds in the targeted treatment area to allow molecules larger than the normal limit (e.g., greater than 400 Da) to pass through the capillary interface and encounter the soft tissue cells in the targeted treatment area.

The result of such an increase in the permeability of vascularized soft tissue tumors is to dramatically increase the intratumoral concentration of native therapeutics, thereby, decreasing the required systemic therapeutic dose and associated dose dependent side effects. That is, the sonoporation of local capillary beds in the target treatment area may allow for increased permeability with respect to the therapeutic agent, thus increasing the effective delivery of the therapeutic agent to the targeted treatment area. As will be shown below, use of ultrasound imaging PMB products and generic ultrasound systems have demonstrated variable results and have identified factors driving therapeutic heterogeneity. In turn, the present disclosure directly addresses each major cause of treatment heterogeneity to provide a system purpose-built for tumor sonoporation to facilitate the noted benefits while reducing or eliminating the traditional barriers limiting use of microbubbles for sonoporation. In turn, the sonoporation systems of the present disclosure are specifically designed to increase vascular permeability to circulating drugs in tissue, including tumors.

Although the presence of “leaky” capillary vessels in tumor tissue that are permissive to passive or ultrasound driven transport of therapeutic agents is often discussed, by virtue of clinical outcomes alone, is not always sufficient to permit entry of therapeutic agents into tumor tissue. In turn, the therapeutic advantage provided by the present disclosure is temporarily disrupting epithelial cell tight junctions within vascularized soft tissue tumors to increase the diffusion-limited rate of transfer of circulatory therapeutic agents towards direct contact with target tumor tissue. To this end, the present disclosure presents SIMs with tight size distributions and a longer circulatory half-life along with a purpose-built ultrasound delivery and monitoring of the therapy.

In addition to a specifically formulated SIM product, the present disclosure contemplates a sonoporation system including an ultrasound device to uniquely image, treat, and monitor sonoporation of target tissue regions in targeted clinical settings. The sonoporation system uses diagnostic-level ultrasound pressures focused to a user-selectable target region. The sonoporation system of the present disclosure provide a specifically adapted ultrasound device for sonoporation of tumor tissue and for monitoring this sonoporation in real time.

The presently contemplated sonoporation system will use image processing algorithms to allow the user to confirm that the treatment is being successfully applied to the target tissues. This feature may include full 3D monitoring if utilizing CT or MR registration. In addition, the sonoporation system contemplated herein may function in an outpatient setting, and thus not require the patient to engage multiple systems, specialties, practitioners. The system will image and treat soft tissue tumors. Such tumors may be located throughout the human torso, although other tumor locations may also be treated. The sonoporation system may use diagnostic 3D ultrasound to image the tumor and margins to be treated. In turn, the system may provide focused therapeutic ultrasound at specific tumor locations identified from imaging. The system may also inform clinician as to the defined therapeutic margins and monitor the sonoporation in real time. A user interface may provide data as to the degree of sonoporation delivered throughout the targeted tumor.

Sonoporation systems according to the present disclosure may include SIMs that are produced at commercially viable high yields. As noted above, production of microbubbles in a target size range have not been sufficiently effective to provide sufficient yields. In addition, many microbubble products previously contemplated include PMB distributions. In turn, traditional microbubble production approaches have demonstrated an inability to effectively isolate microbubbles at a target size at sufficiently high yields. Thus, while traditional microbubble production may produce relatively high yields of PMB populations, the process to isolate the target size of microbubbles may significantly reduce the resulting yield of the size isolated microbubbles. Moreover, preservation (e.g., during distribution and storage) may be difficult to achieve, thus resulting in a limited shelf life of PMB microbubbles.

While certain examples presented herein relate to use of liposomal doxorubicin (“L-DOX”) as a chemotherapy agent for targeting vascularized soft tissue tumors, other drug types may also be administered using the techniques described herein. This may extend to other types of chemotherapy drugs (e.g., using metronomic dosing) including doxorubicin, topotecan, cyclophosphamide, vincristine, and/or etoposide. Such chemotherapy agents may be delivered in conjunction with SIMs in the size range of 4-5 μm. This may include SIMs in a size range of about 4μm to about 5μm at a volume percent of 60% or greater, 70% or greater, 80% or greater, 90% or greater, or even 95% or greater. In other examples, any application in which a therapeutic agent provided in a patient's bloodstream is desirably targeted to a specific anatomy may employ sonoporation using SIMS as described herein.

In one example, targeted ultrasound may be provided in a targeted treatment area for the treatment of relapsed and high-risk neuroblastoma. However, other applications in which drugs are desired to be targeted as vascularized soft tissue are contemplated without limitation. The resulting sonoporation increases the permeability of tissue (e.g., tumor tissue) and allows greater infiltration of circulating therapeutic agents (e.g., chemotherapy agents) into the tumor tissue. This enables more effective delivery of therapy at approved doses, leading to increased efficacy. Furthermore, in some examples, use of sonoporation as described herein with SIMs allows a reduction in dose compared to an approved therapy dose of an agent, thus leading to a reduction in systemic side-effects while maintaining current therapy efficacy. In some examples, administration rates of agents with sonoporation in the presence of size isolated microbubbles may allow administration rates at 50% or less, 40% or less, 30% or less, or even 20% or less of approved administration rates in the absence of sonoporation.

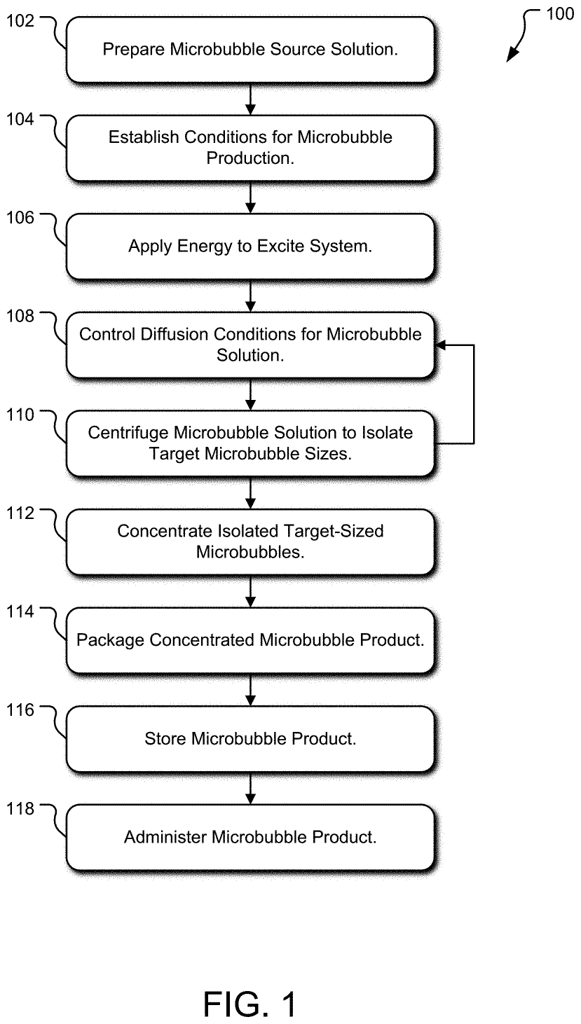

Accordingly, the sonoporation system of the present disclosure includes a SIM product produced at high yields. FIG. 1 includes a flow-chart that depicts an embodiment of a method 100 for production of SIMS at high-yield to produce a SIM product. The method 100 may include preparing 102 a microbubble source solution. The source solution may be a combination of components from which microbubbles are initially formed to produce a polydisperse population of microbubbles. In an embodiment, this may include a lipid suspension for use in preparing lipid shelled microbubbles. The preparing 102 may include preparing a solution comprising a mixture of lipid phosphatidylcholine (PC) and a lipid with a polyethylene glycol (PEG) group. The ratio of PC to PEG may be at a 9:1 molar ratio. While any appropriate lipid may be utilized, the PC lipid may range from a chain length of 14-24. In other embodiments, headgroup modified lipids containing chemically bound groups, such as groups bound by covalent bonding or streptavidin-avidin linkage, may also be used in the solution. The PEG molecular weight may range from 2000-5000. The lipid components may be combined in an aqueous medium. In an embodiment, the aqueous medium may be phosphate buffered saline (PBS). In any regard, the lipid component may be mixed with the aqueous medium to provide a lipid solution. Other components may be added such as surfactants (e.g., polymeric surfactants) or proteins (e.g., albumin), which may provide more stable microbubbles. Further stabilizing agents or stabilizers may be added to the lipid solution as well. For instance, one or more stabilizers including a fatty acid, dextrose, medical grade honey, lecithin, pectin, xanthan gum, cholesterol, casein, gum Arabic, and/or fatty alcohols may be added, which may promote stability of resulting microbubbles as described in PCT Publication No. WO 2021/141595 published on 15 Jul. 2021, the entirety of which is incorporated herein in its entirety.

The method 100 may include establishing 104 conditions for microbubble generation. This may include heating the lipid solution to above the phase transition temperature of the lipid solution to promote mixing of the lipid components in the aqueous medium. By heating the lipid solution to above the phase transition temperature, the lipid solution may be more solvent relative to the aqueous medium to promote mixing. Furthermore, the mixture may also be physically mixed while heated. The lipid solution may be further excited to break up large lipid aggregates into smaller micelles and liposomes. This may include applying energy using an energy applicator such as a sonicator or the like. The energy applicator may be the same as an energy applicator discussed below for excitation to produce microbubbles. However, the energy applicator may be operated at a lower power setting to promote mixing of the lipid component in the aqueous medium prior to generation of microbubbles. In any regard, the suspension may be energized (e.g., sonicated, agitated, or otherwise physically excited) until the solution is translucent and there are no visible aggregates. Once the mixture is translucent and no visible aggregates are present, the lipid solution may be cooled to below the phase transition temperature of the lipid component of the lipid solution. For instance, the temperature of the lipid solution may be set to no less than 5° C. below the phase transition temperature of the main phospholipid in the solution.

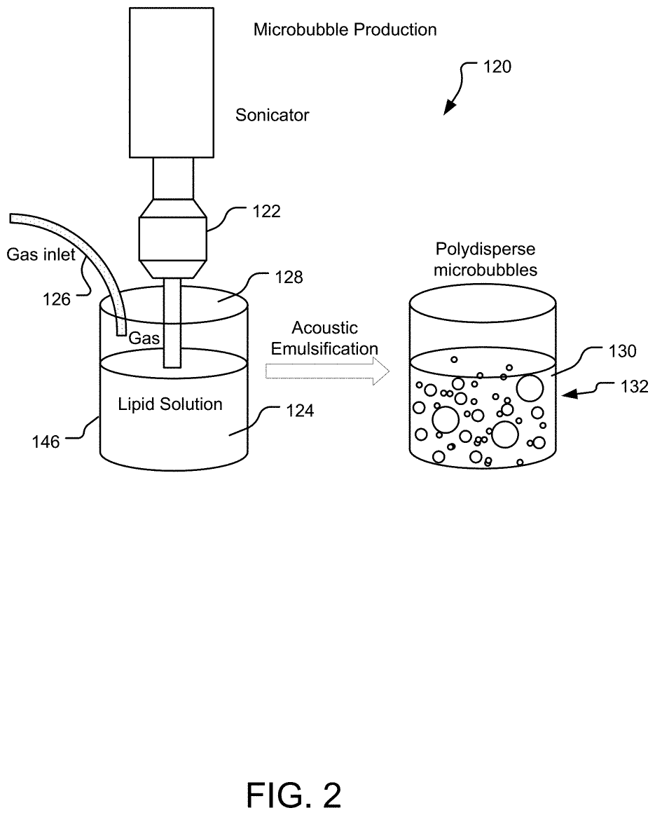

The establishing 104 of the conditions for microbubble production may also include introduction of a gas phase into a headspace of a vessel containing the lipid solution. With further reference to FIG. 2, an embodiment of a system 120 that may be utilized in relation to microbubble production is depicted. In this regard, the lipid solution 124 as described above may be provided according to the foregoing. The lipid solution 124 may be disposed in a vessel 146. In turn, a gas inlet 126 may be introduced into a headspace 128 of the vessel 146. In turn, a gas may be introduced into the headspace 128 at a pressure greater than atmospheric. Initially, the gas may be used to flush out or purge ambient air in the headspace 128. Thereafter, the headspace 128 may be maintained at an elevated pressure (e.g., to provide a slight indentation on the surface of the lipid suspension 124). The gas introduced in the headspace 120 may be any appropriate gas, but at least in one embodiment the gas may be perfluorobutane or other high molecular weight gas.

The method 100 may further include applying 106 energy to excite the system and create microbubbles. As described above, this may include physical agitation of the lipid solution 124. With returned reference to FIG. 2, a sonicator 122 may be introduced such that the sonicator 122 is disposed at the interface between the pressurized gas in the headspace 128 and the lipid solution 124 in the vessel 146. In turn, the sonicator 122 may be activated to sonicate the interface and create the microbubbles through mechanical agitation (e.g., acoustic emulsification) on of the interface between the high-pressure gas and the lipid solution 124. While a sonicator 122 is shown and discussed, other mechanisms for physical agitation of the interface between the high-pressure gas and the lipid solution 124 may be utilized without limitation such as a mechanical agitator for shaking the lipid solution 124, a dental amalgamator for acting on the lipid solution 124, or an in-line homogenizer/colloid mill for acting on the lipid solution 124.

It has been found that the initial microbubble yield may be increased by exciting a relatively warm lipid solution 124. In this regard, the warm lipid solution may be maintained below the main lipid phase transition temperature of the solution but maintained relatively close to the phase transition temperature (e.g., within 5° C. of the main lipid phase transition temperature of the solution). In this regard, the lipid solution 124 may be disposed relative to a thermal regulation device such as a heater and/or cooler to maintain the temperature of the lipid solution 124.

Once initial microbubble production has been completed, the method 100 may include controlling 108 one or more diffusion parameters to maintain preferable diffusion conditions for the microbubble suspension 132. The microbubble suspension 132 may be maintained in the aqueous solution of the lipid solution 124 or may be collected and transferred to a virgin aqueous solution. Further still, the microbubble suspension 132 may be centrifuged to collect the polydisperse microbubble population in a supernatant cake for collection. In any regard, further details regarding the controlling 108 of the diffusion parameters is described in greater detail below. However, the controlling 108 may include establishing conditions in which the diffusion forces acting on the microbubbles in a solution are maintained within a predetermined range to reduce or minimize the diffusion forces in the microbubble suspension 132. Specifically, it is noted that to provide increased yield of lipid stabilized, size isolated microbubbles using differential centrifugation, it is important to optimize initial bubble production while minimizing factors the results in microbubble gas dissolution and breakdown of microbubbles during centrifugation wash cycles of the size isolation process. In this regard, it is presently recognized that it is possible to reduce or impede gas dissolution of the microbubble by minimizing the diffusion force that governs bubble the solution. Specifically, the Stokes-Einstein's Brownian diffusion equation for gas dissolution provides characterization of the gas dissolution of a system comprising microbubbles with a diffusion constant D. The Stokes-Einstein's Brownian diffusion equation can be represented as:

$$D = \frac{kT}{6 {πμ}^{*} r} \qquad Equation 1$$

where k is the gas constant, T as the temperature of the ambient fluid, μ* as the effective viscosity of the fluid, and r in the radius of the microbubble. In addition, it is recognized that the effective viscosity of the fluid medium is at least in part based upon the viscosity of the fluid containing the microbubbles and the volume fraction of the microbubbles in the fluid. Specifically, a model to determine the effective fluid viscosity in view of crowding and bubble interaction is provided as:

$$\frac{{μ}^{*}}{μ} = 1 + 2.5 Φ + 7.6 {Φ}^{2} \qquad Equation 2$$

where μ* is the effective viscosity of the solution, p is the viscosity of the fluid in which the microbubbles are provided, and c as the bubble volume fraction. As will be described in greater detail below, any of the parameters capable of being controlled in relation to the system from Equation 1 may be controlled 108 to reduce or minimize the diffusion forces acting on the microbubbles in the microbubble solution 132.

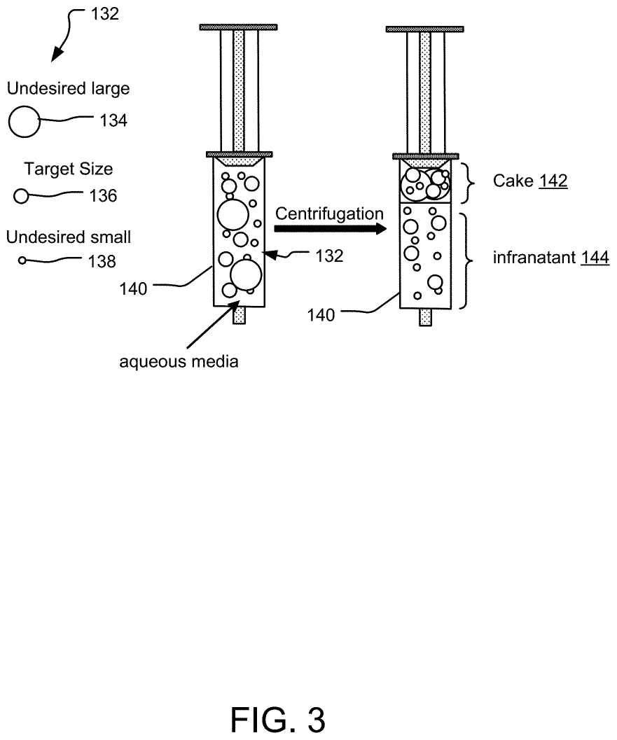

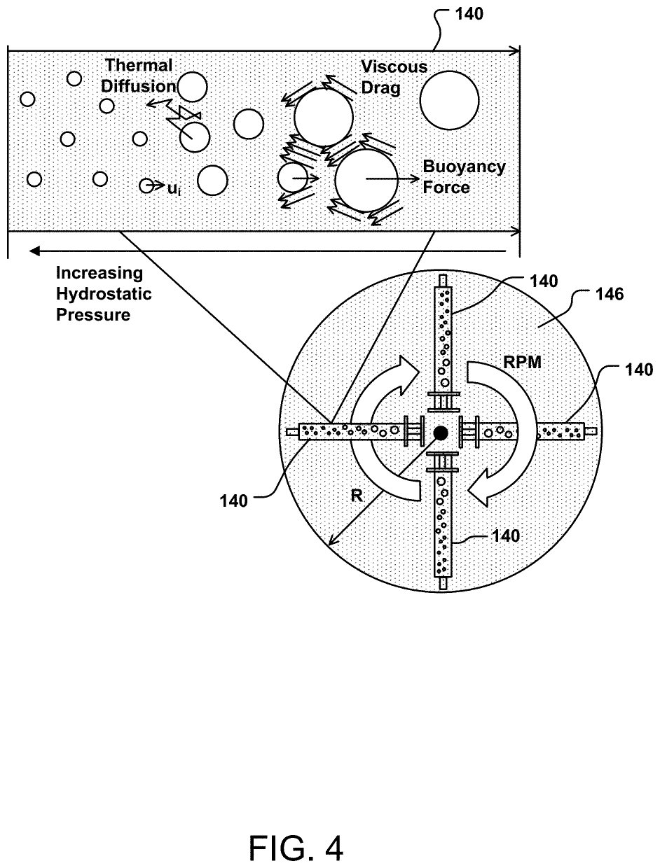

In any regard, the method 100 may include centrifuging 110 the microbubble solution to isolated target microbubble sizes. This may include a multiple step centrifugal wash cycle in which the solution may be subjected to a number of centrifuging washes to isolate a given target size microbubble. With reference to FIG. 3, one example of a wash cycle is illustrated. As can be appreciated, a separation column 140 may contain a microbubble solution 132 that includes undesired large microbubbles 134, target sized microbubbles 136, and undesired small microbubbles 138. With additional reference to FIG. 4, a detailed view of a separation column 140 in a centrifuge 146 is shown. As can be appreciated, upon application of a relative centrifugal force (RCF) to the separation column 140, differential centrifugation may occur to effectively separate the polydisperse population of microbubbles based on size in view of the forces acting on the microbubbles as shown in FIG. 4.

The separation column 140 may be subjected to centrifugation to produce a supernatant cake or simply cake 142 and an infranatant 144. In this regard, a number of wash cycles may be applied to selectively isolate various sized microbubbles in the cake 142 or infranatant 144, depending on the wash cycle. For example, the centrifuging 110 may include applying a first centrifugal field having a first field strength to a suspension comprising a polydisperse population of microbubbles for a first duration of time, thereby forming a first infranatant 144 comprising at least a portion of target microbubbles and a first cake 142 comprising microbubbles having a greater size than the target microbubbles. In turn, the first cake 142 may be removed. In turn, the first infranatant 144 may be applied thereto a second centrifugal field having a second field strength to the first infranatant for a second duration of time, the second field strength to form a second supernatant cake 142 comprising at least a portion of the target microbubbles and second infranatant 144 comprising microbubbles having a smaller size than the target microbubbles. Thus, the second supernatant cake 142 that contains the target size microbubbles may be isolated.

In this regard, it may be appreciated that because the diffusion coefficient that describes the diffusion forces acting on a microbubble in the microbubble solution 132 may be at least in part based on the size (e.g., radius) of the microbubble, the controlling 108 of the diffusion factors may be based on the target size of the microbubble targeted in the centrifuging 110. In any regard, once isolated, the target size microbubbles may be concentrated 112.

As will be discussed in greater detail below, by facilitating high-yield size isolated microbubble populations, a relatively large volume of highly concentrated size isolated microbubbles may be realized. In this regard, the resulting size isolated microbubble product may provide very high-volume fractions in a resultant solution. As can be appreciated from the foregoing equations, such a high-volume fraction may result in a high effective viscosity of a final size isolated microbubble product. This may result in a low diffusion coefficient, indicative of low diffusion forces in the concentrated size isolated microbubble product. Accordingly, the resulting high-yield, size isolated microbubble product may demonstrate improved stability as the diffusion forces associated with microbubble gas dissolution and breakdown may be minimized.

As such, the resulting microbubble product may be packaged 114 in an appropriate form. Any suitable vessel may be utilized for containment of the packaged 114 concentrated microbubble product. However, certain product forms are specifically contemplated herein such as a vial or syringe to contain the concentrated microbubble product. Advantageously, when contained in a syringe, the resulting concentrated microbubble product may be injectable without further handling of the microbubbles. By minimizing the handling or transfer of the size isolated microbubbles, the high-yields of the foregoing process may be maintained with minimal degradation of the microbubbles due to transfer between vessels, which may result in breakdown or other degradation of the microbubbles. In an embodiment, the internal volume of the vessel into which the concentrated microbubble product is provided may be the same as the volume of the concentrated microbubble product itself. That is, the container into which the concentrated microbubble product is provided may have substantially no headspace such that the concentrated microbubble product occupies substantially all of the internal volume of the vessel. This may also assist in reduction of the dissolution forces that tend to break down microbubbles during storage.

As such, the improved stability of the concentrated microbubble product may allow for storage 116 of the microbubble product. The storage 116 may be at room temperature or may be at a refrigerated temperature to promote microbubble preservation. For instance, when stored at a refrigerated temperature (e.g., not greater than 4° C.), at least 90% of the initial volume of the concentrated microbubble product disposed in the containment vessel during the packaging 114 may remain after not less than 4 weeks of storage 116. In addition, when stored at room temperature (e.g., not greater than 23° C.), at least 80% of the initial volume of the concentrated microbubble product disposed in the containment vessel during the packaging 114 may remain after not less than 4 weeks storage. As may be appreciated, the increased stability of the microbubble product may allow for enhanced shelf life, thus facilitating maintaining the microbubble product in inventory at a medical facility prior to use. This may allow for increased flexibility in relation to the use of the microbubble product for various medical applications such as use as a contrast agent in medical imaging, ultrasound targeted drug delivery, ultrasound targeted ablation of tissue, ultrasound aided opening of tissue or any other medical application in which size isolated microbubbles may be utilized. Thus, the method 100 may further include administering 118 the microbubble product and a medical application.

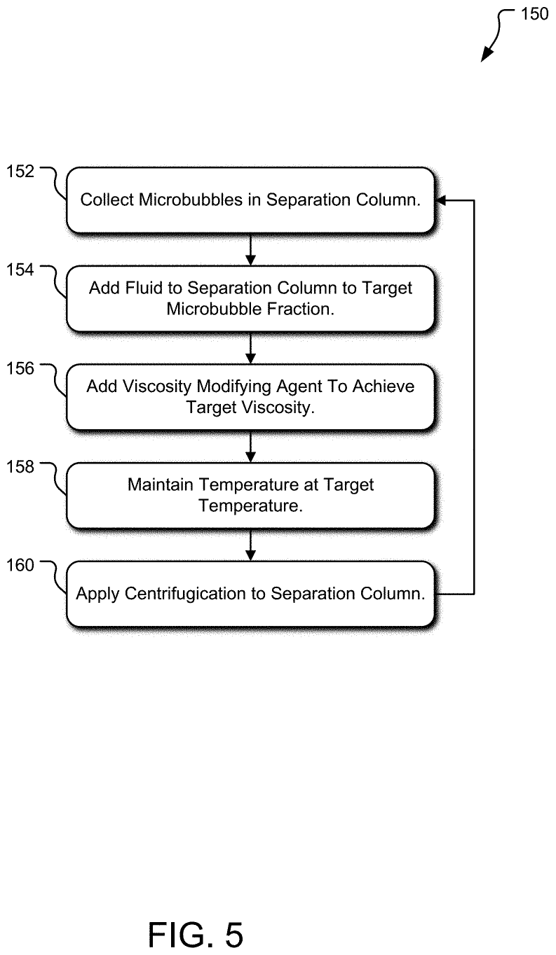

With further reference to FIG. 5, a method 150 for controlling one or more diffusion factors for a microbubble solution 132 during application of centrifugation for size isolation is shown. The method 150 may include collecting 152 microbubbles into separation column 140. As shown in FIG. 3, the separation column may be a syringe, which may also be used to store 116 a resulting size isolated microbubble product. The method 150 may further include adding 154 fluid to the separation column to achieve a target microbubble fraction. As discussed above, increases in the target microbubble fraction may result in reduction in the diffusion coefficient, and thus the diffusion forces acting on the microbubbles in the separation column during application of centrifugation for size isolation. In an embodiment, the target microbubble fraction may be not less than about 20% and not greater than about 50%. However, in other approaches the target microbubble fraction may be not less than 25%, not less than 30%, or not less than 40%. Further still, the target microbubble fraction may be not greater than about 45%, not greater than about 40%, or not greater than about 30%. Furthermore, various target microbubble fraction ranges may be provided according to any of the minimum and maximum values listed above, provided that the maximum value is greater than the minimum value of the range. In this regard, by maintaining a microbubble fraction at least about 20%, the effective viscosity of the solution may be increased, thus decreasing the diffusion forces per Equation 1 above.

In addition, while microbubble fraction is recognized as affecting the effective viscosity of the solution, the viscosity of the fluid in which the microbubbles are contained is also a factor effective viscosity. In this regard, the viscosity of the fluid in which the microbubbles are provided may also be controlled (e.g., to increase the viscosity of the solution and thus reduce the diffusion coefficient of the system). In this regard, a viscosity modifier may be added 156 to the microbubble solution to target effective viscosity. In an embodiment, the viscosity modifier may comprise glycerol.

Furthermore, the temperature of the solution may also affect the diffusion coefficient governing the diffusion forces acting on the microbubbles in the solution. As such, the method 150 may include maintaining 158 a temperature of the solution at a target temperature. As described above, during initial microbubble production, the lipid solution from which the microbubbles are produced may be preferably maintained at below about 5° C. less than the phase transition temperature of the lipid solution. In this regard, the solution during initial microbubble production may be at a relatively elevated temperature. As such, the maintaining 158 of the temperature of the solution may include reducing the temperature of the solution to well below that of the temperature at which the microbubbles are initially produced from the lipid solution. This may include extraction of the microbubbles from the lipid solution remaining after initial bubble production. In this regard, the extracted microbubbles may be refrigerated or may be introduced into a fluid (e.g., during adding 154 of the fluid to the separation column) that is at a refrigerated temperature. For instance, the target temperature of the solution during centrifugation for size isolation may be not greater than about 1° C., not greater than about 4° C., not greater than about 5° C., not greater than about 10° C., or not greater than about 15° C.

The method 150 may also include applying 160 centrifugation to the separation column for size isolation of a target size microbubble. The applying 160 may be performed in view of the prior steps of the method 150 that effectively control the diffusion parameters of the system. As may be appreciated in FIG. 5, the method 150 may iterate such that after application 160 of centrifugation to the separation column, the method 150 may repeat. In this regard, and each successive iteration of the method 150, the control of the various diffusion parameters provided in the steps of the method 150 may be altered based upon the particular centrifugal washing being applied. For instance, if different sized microbubbles are targeted and subsequent washes, the diffusion parameters maintained may vary.

The use of SIMS in the presently contemplated sonoporation system is important for a number of reasons. Such a SIM product may carry a pre-formed stabilized gas bubble that can create sustained levels of stable cavitation (bubble expansion and collapse) when exposed to ultrasound energy at the site of a tumor. This purely mechanical effect is used to temporarily disrupt epithelial tight junctions within the capillary bed of soft tissue tumors.

Accordingly, in the sonoporation system contemplated herein, SIMs may be co-administered independently alongside a therapeutic agent (e.g., a native anti-cancer agent). Specifically, SIMs in a size of 4-5 μm may be used as defined above. By virtue of their size, the SIMs circulate in the blood stream and, when exposed to diagnostic ultrasound focused at specific regions of a tumor, temporarily disrupt endothelial tight junctions by popping or cavitating. This may increase permeability to therapeutic agents within solid tumors. Preclinical data supports increased penetration of therapeutic agents into tumor tissue and a concurrent decrease in systemic levels of therapeutic agents needed to produce a therapeutic effect.

SIMs exhibit a number of enabling features that provide specific clinical advantages to the present contemplated sonoporation system. For example, the SIMs provided noted benefits on tissue effects. Specifically, the SIM particles are designed to provide specific, reproducible tissue effects focused on temporary disruption of epithelial tight junctions. SIMs also provide advantages in pharmacokinetics because the in vivo circulation kinetics of SIMS have been designed to be compatible with a broad range, if not all, therapeutic oncology agents. The SIMs also provide sufficient time to administer ultrasound to the anatomic region of interest. In relation to administration, SIMS provide advantages as the SIMs are designed to adopt the same administration route as the therapeutic agent, including intravenous (IV) or intra-tumoral (IT). In addition, SIMs may be independently administered alongside the therapeutic agent such that no drug reformulation is required. In turn, the SIMs fit neatly within current oncology ward out-patient clinical practice. That is, for intravenous administration, the SIMs will be delivered to the patient via an infusion bag and giving set making their administration fit current practice without requiring practitioners to be trained for new administration practices specific to the sonoporation system presented herein.

Furthermore, SIMs may address a number of therapeutic variables that, as noted above, have limited use of prior PMB agents that exhibit a polydisperse population of bubbles. Specifically, PMBs may result in a number of therapeutic heterogeneities that limit the clinical efficacy of sonoporation. The noted therapeutic benefits are reflected in Table 1 below:

THERAPEUTIC VARIABLES ADDRESSED

| Cause of Heterogeneity | Effect | Mitigation Strategy |
|---|---|---|
| Bubbles <2 μM | Little to no sonoperforation | Eliminate bubbles <3 μM |
| | using moderate ultrasound | from microbubble |
| | energy levels, high energy | formulation. |
| | required risks tissue damage | |
| | [28]. | |
| Bubbles >8 μM | Rapid translational motion, | Eliminate bubbles >6 μM |
| | too much energy delivered, | from formulation. |
| | killing cells [28]. | |
| Short circulatory half-life: for | Very brief timing windows | SIMs provide a circulatory |
| sonoperforation to occur, | when the concentration of | half-live >3X greater than |
| distance from target must be | bubbles within the therapeutic | marketed image |
| less than about the diameter of | distance limits efficacy. | enhancement microbubbles. |
| bubble. Rapid variance in | | |
| circulatory concentration | | |
| directly varies the therapeutic | | |
| concentration. | | |
| Unvalidated sonoporation | Adapting non-validated, off- | A purpose-built ultrasound |
| systems | label products to sonoporate | delivery and therapy |
| | tumor tissue should not be | monitoring system, |
| | relied upon for a broadly | clinically validated and |
| | adopted therapeutic for life- | optimized for therapeutic |
| | threatening conditions. | efficacy. |
| Naïve Tumor Capillary | Capillary permeably within | Utilize sonoporation to |
| Permeability | tumors is variable, relying on | temporarily perforate |
| | native capillary structures for | capillary calls ensuring |
| | therapeutic efficacy causes | chemotherapeutic agent |
| | variability. | penetration into tumor |
| | | tissue. |

Accordingly, the sonoporation system of the present disclosure may advantageously use a minimum amount of force required to temporarily disrupt epithelial cell tight junctions within vascularized soft tissue tumors to increase the diffusion limited rate of transfer of circulatory therapeutic agents towards direct contact with target tumor tissue. To this end, the use of with limited size distributions provide a longer circulatory half-life. As such, SIMs used along with a purpose-built ultrasound delivery and monitoring system facilitates reductions in the heterogeneity demonstrated by prior approaches and improves the clinical efficacy as demonstrated below.

The monodisperse SIMS significantly improved liposomal doxorubicin (“L-DOX”) uptake in the tumor versus L-DOX alone or administration of L-DOX with the use of unsorted (polydisperse) conventional imaging bubbles. In addition, L-DOX delivery covered the tumor in a homogeneous manner in contrast to uptake using conventional imaging PMBs. This treatment demonstrated efficacy after delivery using SIMS and a low dose of a chemotherapy agent. Importantly, the treatment produced preliminary longitudinal efficacy data demonstrating halting or reduction of tumor volume versus controls and versus a regimen in which a drug was administered alone without sonoporation. In studies using mice, the halting of tumor growth was at a very low dose (e.g., 20-fold less than started) of L-DOX without the mice losing weight or exhibiting other symptoms characteristic of L-DOX side effects. The mice appeared healthy after treatment as opposed to the use of higher doses (25 mg/kg) that caused the mice to lose weight or exhibit other symptoms indicative of side effects of the chemotherapy agent.

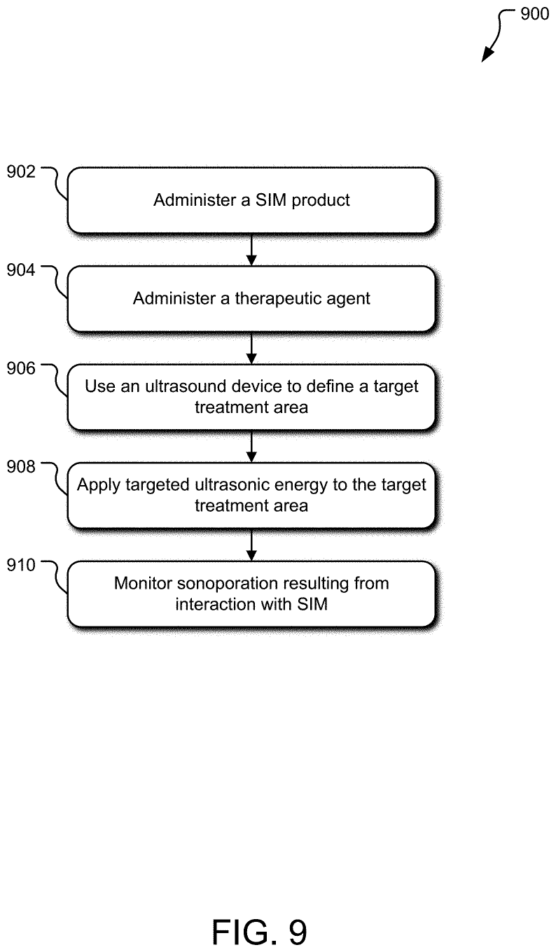

Accordingly, an example set of operations 900 related to a sonoporation system are shown in FIG. 9. The operations 900 include an administering operation 902 in which a SIM product is administered to a patient. As noted above, the administering operation 902 may include intravenous (IV) administration of the SIM product or may include direct injection of the SIM product into soft tissue to be targeted. The operations 900 may also include an administering operation 904 which a therapeutic agent is also administered to a patient. Like the SIM product, the administering operation 904 may include intravenous (IV) administration or direct injection into soft tissue to be targeted. In addition, administering operation 904 may include administering the therapeutic agent at a significantly reduced dosage as compared to an approved dosage of the therapeutic agent as noted above.

The operations 900 may also include an imaging operation 906 in which an ultrasound device may be used to define a target treatment area. Specifically, definition of a target treatment area may include imaging a soft tissue target to which therapeutic agent is targeted. The imaging operation 906 may include determining margins of a tumor to assist in monitoring the efficacy and/or status of therapy applied to the soft tissue target. In addition, the operations 900 may include a targeting operation 908 in which targeted ultrasonic energy is targeted to the treatment area. The targeting operation 908 may include application of targeted ultrasonic energy using the same ultrasound device used in the imaging operation 906. In this regard, the imaging operation 906 and targeting operation 908 may be conducted simultaneously or at least in an overlapping time period using the same ultrasound device. In addition, the operations 900 may include a monitoring operation 910 in which the ultrasound device may be used to monitor sonoporation resulting from interaction of the targeted ultrasonic energy with the SIM product in the targeted treatment area. The monitoring operation 910 may include quantification of sonoporation, which may assist in determining efficacy of treatment and may provide feedback to help provide guidance for treatment and/or outcome results of the treatment.

### EXAMPLES

### Example 1: Size Isolated Microbubble Process Summary

Polydisperse microbubbles are prepared by excitation methods such as acoustic emulsification. In the first stage of size-isolation, bubbles below an undesired size are extracted from the polydisperse suspension of high volume fraction (20-30%) using a large capacity column. Since, the bubble population extracted will be at a lower concentration (and lower volume fraction) than the initial population, subsequent separation to remove the undesired smaller sizes is performed by adjusting bubble volume fraction and or fluid viscosity in the next separation column. All centrifugal washing is done with cold aqueous media to control temperature.

Preparation Of The Lipid Suspension
1. 1 to 5 mg/mL lipid suspension is prepared by weighing a mixture of main lipid phosphatidylcholine (PC) and lipid containing a polyethylene glycol (PEG) group at a 9:1 molar ratio. The PC lipid can range from a chain length of 14-24. The PEG molecular weight can range from 2000-5000.
2. The mixture is heated (under mixing) to above the phase transition temperature of the PC lipid.
3. The lipid mixture is further sonicated to break up large lipid aggregates into smaller micelles and liposomes by immersing the tip of a Branson Sonifier® 250/450 probe sonicator into the middle of the suspension and applying half the power output until the solution is translucent and there are no visible aggregates.
4. The lipid mixture is set to at least 5° C. below the phase transition temperature of the main phospholipid.

Polydisperse Microbubble Production at High yield
1. The gas phase (perfluorobutane or high molecular weight gas) is introduced to the headspace at an overpressure that flushes out ambient air and causes a greater than about 0.5 inch indentation on the surface of the suspension.
2. The microbubbles are prepared by acoustic emulsification (probe sonication) by sonicating a full power at the interface of the gas and the warm lipid suspension. Results demonstrate that the initial bubble yield increases dramatically when a warm lipid solution (below the main lipid phase transition temperature) is sonicated during production as opposed to a cold one.
3. Microbubbles are carefully collected and drawn into large columns (30 mL or 60 mL) with care taken not to collect undesirable the macroscopic bubble foam residue.
4. The syringe columns are centrifuged at higher field force to collect all microbubbles in cake (top phase) and non-gas microbubble particles such as liposomes and micelles into infranatant (bottom phase). For example, 300 RCF for 5 minutes is used in 30 mL column to collect all microbubbles. The concentrated polydisperse microbubbles are collected and infranatant is discarded or recycled.

Stage 1: Extracting Target Sizes and Small Sizes from Undesired Large Sizes Using Column at High Bubble Fraction and Cold Media:
1. The microbubbles cakes produced are combined into a separation column (30 mL or 60 mL) by diluting the cakes and combining them into a separation column using cold aqueous media (i.e., pH 7.4 saline). The resulting suspension is diluted to a volume fraction of 20-30%.
2. Lower strength field is applied to cause all bubbles of undesired large size and above to collect into the cake.
3. The infranatant containing few or substantially none of the undesired large sizes and mixture of desired size target and undesired small are collected, concentrated and then transferred to another separation column via syringe-to-syringe transfer.

Stage 2: Removing Undesired Small Sizes from Target Sizes While Maintaining High Bubble Fraction and Washing with Cold Media:
1. The column contents are carefully adjusted to 20-30% volume fraction of microbubbles with cold aqueous media in order to maintain the high-volume fraction/high fluid viscosity and low temperature.
2. High field centrifugal force is applied to cause the target size to form into the cake and the undesired small sizes to remain in the infranatant.
3. Due to the nature of greater abundance of the smaller undesirable sizes, the cake is reconstituted with the cold aqueous media and the high field centrifugal force is applied in multiple cycles to remove undesired small sizes from the target size.
4. The final target size is concentrated and transferred to storage container.

This method may allow for the production of four different size classes at high yield including 2 micron bubbles at a yield of greater than 2×10¹⁰ MB/mL, 3 micron bubbles at a yield of 1×10¹⁰ MB/mL, 4 micron bubbles at a yield of 8×10⁹ MB/mL and 6 micron bubbles at a yield of 2×10⁹ MB/mL.

Effect Of Bubble Fraction and Temperature on Yield of Size-Isolated Microbubbles
a. Maintaining High Bubble Fraction Versus Reducing Bubble Fraction—Cold Temperature Washing
   The table below provides a comparison of the yield of size sorted bubbles for maintaining a high bubble (25%) fraction/high viscosity versus reducing the bubble fraction from high (25%) to low (6%) in the washing stages. The same bubble production method is used for both test columns and cold aqueous media is adopted for centrifugal washing. The cake yield for maintaining a high bubble fraction (25%) is approximately 4-fold greater than reducing bubble fraction to 6%. The microbubble yield for maintaining a high bubble (25%) fraction is approximately 10-fold greater than reducing bubble fraction to 6%.

Comparison Of Size-Isolation by Maintaining High Bubble Fraction Vs
Lowering Bubble Fraction at Same T

| | High (25%) to high (25%) | High (25%) to low (6%) |
|---|---|---|
| | bubble fraction | bubble fraction |
| Cake Yield | 2 mL | 0.5 mL |
| Microbubble yield | 1e10 microbubbles | 1e9 microbubbles |

b. Cold (5° C.) Vs Room Temperature (22° C.) Aqueous Media At High Bubble Fraction
   The table below provides a comparison of the yield of size sorted bubbles when adopting aqueous media at temperature of 5° C. versus 22° C. and maintaining a high bubble fraction (25%) during differential centrifugation. The cake yield for 5° C. is ˜2 fold greater than 22° C. The microbubble yield for 5° C. is approximately 3-fold greater than 22° C.

Comparison of Size-Isolation using Cold vs Warm media while maintaining
high bubble fraction

| | 5° C. media | 22° C. media |
|---|---|---|
| Cake Yield | 2 mL | 1 mL |
| Microbubble yield | 1e10 microbubbles | 3.0e9 microbubbles |

Example Values for Diffusion Coefficients for High Yield Processing

The following represents example diffusion coefficients for a number of potential conditions for high yield microbubble isolation. Throughout the following, the following, a Boltzmann's constant of k=1.38E−23 J/K is presumed.

In one example, water is used as a fluid medium in which the microbubbles are provided. As such, viscosity values at various temperatures are provided as follows:
μ(water) at 1° C.=0.00173 Pa·s
μ(water) at 25° C.=0.00089 Pa·s

As such, diffusion coefficients based on microbubble diameter for a bubble fraction of =5% are provided when the effective viscosity is μ*(5%, 1° C.)=0.00197912 Pa·s, the values being:

| Diameter (μm) | Diffusion Constant (m²/s) |
|---|---|
| 2 | 1.01E−13 |
| 4 | 5.07E−14 |
| 6 | 3.38E−14 |
| 8 | 2.54E−14 |

With water at an elevated temperature of 25° C., the effective viscosity is μ*(5% , 25° C.)=0.00101816 Pa·s, and the diffusion equation values based on target microbubble size are:

| Diameter (μm) | Diffusion Constant (m²/s) |
|---|---|
| 2 | 2.14E−13 |
| 4 | 1.07E−13 |
| 6 | 7.15E−14 |
| 8 | 5.36E−14 |

Diffusion coefficients based on microbubble diameter for a bubble fraction of Φ=25% are provided such that the effective viscosity is μ*(25%, 1° C.)=0.003633 Pa·s, the values being:

| Diameter | Diffusion |
|---|---|
| (μm) | Constant (m²/s) |
| 2 | 5.52E−14 |
| 4 | 2.76E−14 |
| 6 | 1.84E−14 |
| 8 | 1.38E−14 |

With water at an elevated temperature of 25° C., the effective viscosity is μ*(25%, 25° C.)=0.001869 Pa·s, and the diffusion constant values based on target microbubble size are:

| Diameter | Diffusion |
|---|---|
| (μm) | Constant (m²/s) |
| 2 | 1.17E−13 |
| 4 | 5.84E−14 |
| 6 | 3.89E−14 |
| 8 | 2.92E−14 |

Diffusion coefficients based on microbubble diameter for a bubble fraction of Φ=50% are provided such that the effective viscosity is μ*(50%, 1° C.)=0.0071795 Pa·s, the values being:

| Diameter | Diffusion |
|---|---|
| (μm) | Constant (m²/s) |
| 2 | 2.80E−14 |
| 4 | 1.40E−14 |
| 6 | 9.32E−15 |
| 8 | 6.99E−15 |

With water at an elevated temperature of 25° C., the effective viscosity is μ*(50%, 25° C.)=0.0036935 Pa·s, and the diffusion constant values based on microbubble size are:

| Diameter | Diffusion |
|---|---|
| (μm) | Constant (m²/s) |
| 2 | 5.91E−14 |
| 4 | 2.95E−14 |
| 6 | 1.97E−14 |
| 8 | 1.48E−14 |

Alternatively, glycerol may be used as a fluid medium in which the microbubbles are provided. As such, viscosity values at various temperatures are provided as follows:
μ(glycerol) at 1° C.=10.693 Pa·s
μ(glycerol) at 25° C.=0.905 Pa·s

Diffusion coefficients based on microbubble diameter for a bubble fraction of Φ=5% are provided such that the effective viscosity is μ*(5%, 1° C.)=12.232792 Pa·s, the values being:

| Diameter (μm) | Diffusion Constant (m²/s) |
|---|---|
| 2 | 1.64E−17 |
| 4 | 8.20E−18 |
| 6 | 5.47E−18 |
| 8 | 4.10E−18 |

With glycerol at an elevated temperature of 25° C., the effective viscosity is μ*(5%, 25° C.)=1.03532 Pa·s, and the diffusion constant values based on target microbubble size are:

| Diameter (μm) | Diffusion Constant (m²/s) |
|---|---|
| 2 | 2.11E−16 |
| 4 | 1.05E−16 |
| 6 | 7.03E−17 |
| 8 | 5.27E−17 |

Diffusion coefficients based on microbubble diameter for a bubble fraction of Φ=25% are provided such that the effective viscosity is μ*(25%, 1° C.)=22.4553 Pa·s, the values being:

| Diameter | Diffusion |
|---|---|
| (μm) | Constant (m²/s) |
| 2 | 8.94E−18 |
| 4 | 4.47E−18 |
| 6 | 2.98E−18 |
| 8 | 2.23E−18 |

With glycerol at an elevated temperature of 25° C., the effective viscosity is μ*(25%, 25° C.)=1.9005 Pa·s, and the diffusion constant values based on target microbubble size are:

| Diameter | Diffusion |
|---|---|
| (μm) | Constant (m²/s) |
| 2 | 1.15E−16 |
| 4 | 5.74E−17 |
| 6 | 3.83E−17 |
| 8 | 2.87E−17 |

Diffusion coefficients based on microbubble diameter for a bubble fraction of Φ=50% are provided such that the effective viscosity is μ*(50%, 1° C.)=44.37595 Pa·s, the values being:

| Diameter | Diffusion |
|---|---|
| (μm) | Constant (m²/s) |
| 2 | 4.52E−18 |
| 4 | 2.26E−18 |
| 6 | 1.51E−18 |
| 8 | 1.13E−18 |

With glycerol at an elevated temperature of 25° C., the effective viscosity is μ*(50%, 25° C.)=3.75575 Pa·s, and the diffusion constant values based on target microbubble size are:

| Diameter | Diffusion |
|---|---|
| (μm) | Constant (m²/s) |
| 2 | 5.81E−17 |
| 4 | 2.91E−17 |
| 6 | 1.94E−17 |
| 8 | 1.45E−17 |

### Example 2: Clinical Results of a Sonoporation System Using SIMS as Comparted to Other Treatment Regimens

The present inventors have preliminary validated the application of SIMs having a size of 4-5 μm as an enhanced drug delivery system in a preclinical model of high-risk Neuroblastoma (NGP-NB). Data show that in presence of sonoporation, 4-5 μm SIMs allow a more homogeneous delivery of a drug (e.g., liposomal doxorubicin (L-DOX)) at a tumor site compared to administration of the drug alone or in combination with other commercial PMB product. Moreover, administration of a low dose of L-DOX (1 mg/kg vs 25 mg/kg) together with 4-5 μm SIMs and sonoporation in NGP-NB tumor-bearing mice achieved tumor growth control over two weeks without observing any side effects traditionally exhibited in traditional L-DOX doses (e.g., 25 mg/kg). On the contrary, the same quantity of L-DOX administered alone was not able to control tumor growth.

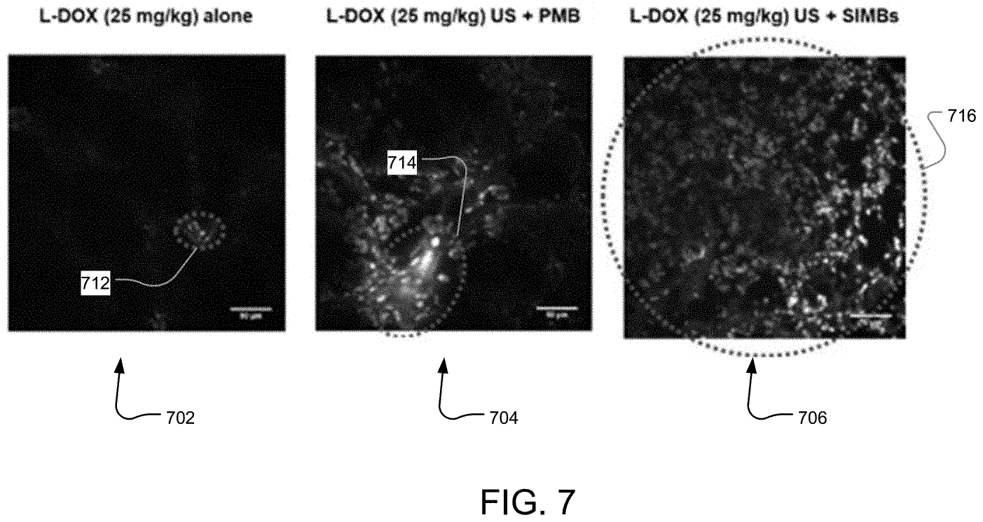

FIG. 7 depicts a comparison of drug uptake within a tumor using the chemotherapy drug L-DOX (25 mg/kg). The image 702 corresponds with results of a 25 mg/kg dose of L-DOX in the absence of any microbubble. Image 704 corresponds with results of a 25 mg/kg dose of L-DOX using sonoporation with a polydisperse microbubble agent. The image 706 corresponds with results of a 25 mg/kg dose of L-DOX using sonoporation with a size isolated microbubble agent having microbubbles in a range of 4-5 μm. As shown in image 702, L-DOX delivery is limited to a treated area 712. In image 704, a treated area 714 is somewhat expanded in view of sonoporation of the polydisperse microbubble agent. However, image 706 illustrates a far larger treated area 716 in which L-DOX is delivered when using a size isolated microbubble product with sonoporation. Even more, the concentration of polydisperse microbubbles administered in image 704 is a ten-time higher concentration as compared to the size isolated microbubble product used in image 706.

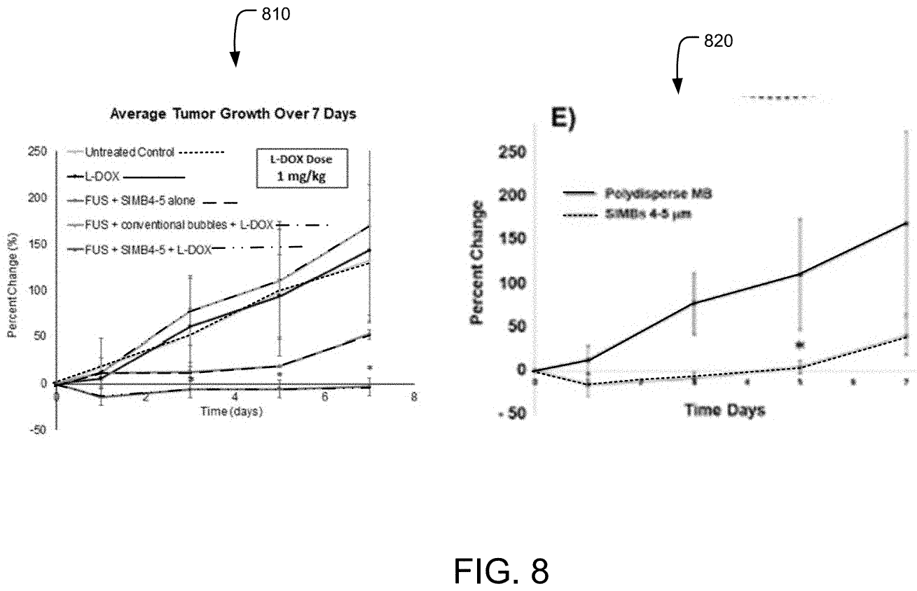

FIG. 8 depicts quantitative results of tumor growth in relation to various treatment regiments. Chart 810 illustrates the effect on tumor growth for four treatment regimens. As can be seen, an untreated control group showed tumor growth of roughly 125%. Similarly, a treatment regimen comprising administration of a dose of L-DOX at 1 mg/kg (a “low dose”) without any sonoporation closely tracked the control, with slightly higher tumor growth values of near 150%. This illustrates that low doses of L-DOX in the absence of sonoporation have little effect on tumor growth as compared to the control. In addition, use of a low dose of L-DOX with a polydisperse microbubble product with sonoporation also demonstrated tumor growth of over 150%. Use of sonoporation with size isolated microbubbles alone (i.e., in the absence of L-DOX) show reduced tumor growth as compared to the control and low dose regimen, yet still shows tumor growth of roughly 50%. In contrast, use of a low dose of L-DOX (e.g., 1 mg/kg) along with sonoporation using size isolated microbubbles demonstrated a reduction in tumor size.

Chart 820 further illustrates the effect on size of microbubble on efficacy of sonoporation using a constant drug dose. As can be seen, polydisperse microbubbles used in sonoporation resulted in a tumor growth of roughly 175% as compared to use of 4-5 μm size isolated microbubbles, which demonstrated tumor growth of less than 50%. Paired T-tests show p-value of 0.3 at day 7 in the results of SIMs alone and SIMS+L-DOX as shown in chart 810 and of 0.045 at day 5 between PMB and SIMS in the chart of 820. As such, the data show that a low dose of the chemotherapy drug L-DOX (1 mg/kg) in combination with sonoporation/SIMS halted tumor growth as compared to L-DOX alone. SIMS allowed a lower and safer dose of L-DOX to be used which resulted in no observable side effects.

These results of tumor growth in relation to treatment using polydisperse sized microbubbles and size isolated microbubbles shows that in the presence of sonoporation, 4-5 μm SIMs allow a more homogeneous delivery of liposomal doxorubicin (L-DOX) at the tumor site compared to administration of the drug alone or in combination with polydisperse microbubbles. Moreover, administration of a low dose of L-DOX (e.g., 1 mg/kg versus a standard dose of 25 mg/kg) together with SIMS having a size of 4-5 μm and sonoporation in NGP-NB tumor-bearing mice achieved tumor growth control over two weeks without observing any side effects. On the contrary, the same quantity of L-DOX administered alone was not able to control tumor growth. To demonstrate SIMs are a more efficient drug delivery system compared to polydisperse microbubbles (PMB), six NGP tumor-bearing mice were divided into two groups treated with low dose L-DOX (1 mg/kg) together with respectively polydisperse microbubbles or 4-5 μm SIMs. On day 0 L-DOX was administered followed by sonoporation with the following parameters 2 MPa peak negative pressure (PNP), 3 W/cm² power and 10% duty cycle. Tumor volumes were measured by caliper every other day until day five, at day seven tumors were measured by 3D imaging. The data shows that low dose L-DOX administration was more efficient in mice sonoporated with SIMS having a diameter of 4-5 μm compared to those treated with PMB.

Accordingly, one example sonoporation system includes SIMS having a size of 4-5 μm. The SIMs may be provided for intravascular administration at a concentration of (2.5×10⁹ microbubbles/mL) followed by insonification of vascularized tumor tissue using a targeted ultrasound sonoporation device. The ultrasound sonoporation device may include an ultrasound transducer equipped with a focusing lens appropriate for a tumor distance from the ultrasound application site using a frequency of 1 MHz and PNP between 0.4-2 MPa (at tumor depth) where temporary permeation by sonoporation of tumor tissue is desired. SIMs of 4-5 μm offer a location selective technique of temporary tumor sonoporation for increasing permeation of therapeutic agents into sonoporated tumor tissues. Ultrasonic energy delivery and site-specific monitoring of sonoporation may also be performed using the sonoporation system.

One general aspect of the present disclosure includes a method for increasing tissue permeation in a target treatment area. The method includes administering to a patient a size-isolated microbubble (SIM) product and introducing ultrasound energy from an ultrasound device to the target treatment area comprising the SIMs. In turn, the method includes sonoporating the target treatment area in response to the ultrasound energy by cavitating the SIMs of the SIM product to increasing tissue permeation the target treatment area by sonoporation of capillary walls of vasculature of the patient in the target treatment area.

Implementations may include one or more of the following features. For example, the method may also include administering a drug to the patient. The increasing tissue permeation in the target treatment area may result in an increased diffusion of the drug from the vasculature of the patient in the target treatment area. In an example, the dose may be at a lower dose than an approved dose of the drug (e.g., a recommended dose for administration in the absence of the sonoporation of the present method). In an example, the dose may be at least half of the approved dose of the drug. In another example, the dose may be at least one tenth of the approved dose of the drug.

In an example, the drug may be a chemotherapy agent. The target treatment area may be a vascularized soft tissue tumor.

In an example, the SIM product may have at least 80 volume percent microbubbles having a diameter of not less than about 4 μm and not more than about 5 μm.

In an example, the method may also include identifying the target treatment area from ultrasound imaging generated by the ultrasound device. Furthermore, the method may include monitoring the sonoporation of the target treatment area using ultrasound imagining generated by the ultrasound device. The identifying and the monitoring may occur in a common time period.

In an example, the sonoporating may include disrupting epithelial cell tight junctions within vascularized soft tissue.

In an example, the administering the SIM product and the administering the dose of the drug may occur in a common injection to the patient.

Another general aspect of the present disclosure includes a sonoporation system for sonoporation of a target treatment area to tissue permeability in the target treatment area. The system includes a size-isolated microbubble (SIM) product comprising at least 80 volume percent microbubbles having a diameter of not less than about 4 um and not more than about 5 μm at a concentration of not less than about 1.0×10⁹ microbubbles/mL. The system also includes an ultrasound device operative to target the target treatment area with targeted ultrasound energy to cause sonoporation in the target treatment area in response to cavitation of the microbubbles of the SIM product.

Implementations may include one or more of the following features. For example, the system may further include a dose of a therapeutic agent having a dose not greater than about 50% of an approved dose of the therapeutic agent. In an example, the SIM product and the dose of the therapeutic agent may be provided in a common administration vessel for simultaneous administration to a patient. The therapeutic agent may be a chemotherapy agent, and the target treatment area may be a vascularized soft tissue tumor.

In an example, the ultrasound device may be operative to identify the target treatment area from ultrasound imaging generated by the ultrasound device. The ultrasound device may be operative to monitor the sonoporation of the target treatment area using ultrasound imagining generated by the ultrasound device.

In an example, the sonoporation in the target treatment area disrupts epithelial cell tight junctions within vascularized soft tissue.

The description of a feature or features in a particular combination do not exclude the inclusion of an additional feature or features in a variation of the particular combination. Processing steps and sequencing are for illustration only, and such illustrations do not exclude inclusion of other steps or other sequencing of steps to an extent not necessarily incompatible. Additional steps may be included between any illustrated processing steps or before or after any illustrated processing step to an extent not necessarily incompatible.

The terms “comprising”, “containing”, “including” and “having”, and grammatical variations of those terms, are intended to be inclusive and nonlimiting in that the use of such terms indicates the presence of a stated condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or any appropriate grammatical variation of such narrower terms). For example, a statement that something “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. As used herein, a range for a feature refers to one or more values for that feature within an upper limit and lower limit, inclusive of the upper and lower limits, and includes situations in which the upper limit and the lower limit are the same, that is when the range includes a single value represented by the equal upper and lower limits.

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that only the preferred embodiment and variants thereof have been shown and described and that all changes and modifications that come within the spirit of the invention are desired to be protected.

What is claimed is:

1. A method for increasing tissue permeability in a target treatment area, comprising:
administering to a patient a size-isolated microbubble (SIM) product;
introducing ultrasound energy from an ultrasound device to the target treatment area comprising the SIMs; and
sonoporating the target treatment area in response to the ultrasound energy by cavitating the SIMS of the SIM product to increasing tissue permeation the target treatment area by sonoporation of capillary walls of vasculature of the patient in the target treatment area.

2. The method of claim 1, further comprising:
administering a drug to the patient; and
wherein the increasing tissue permeation in the target treatment area results in an increased diffusion of the drug from the vasculature of the patient in the target treatment area.

3. The method of claim 2, wherein the dose is at a lower dose than an approved dose of the drug.

4. The method of claim 3, wherein the dose is at least half of the approved dose of the drug.

5. The method of claim 4, wherein the dose is at least one tenth of the approved dose of the drug.

6. The method of claim 2, wherein the drug comprises a chemotherapy agent, and wherein the target treatment area comprises a vascularized soft tissue tumor.

7. The method of claim 1, wherein the SIM product comprises at least 80 volume percent microbubbles having a diameter of not less than about 4 μm and not more than about 5 μm.

8. The method of claim 1, further comprising:
identifying the target treatment area from ultrasound imaging generated by the ultrasound device.

9. The method of claim 8, further comprising:
monitoring the sonoporation of the target treatment area using ultrasound imagining generated by the ultrasound device.

10. The method of claim 9, wherein the identifying and the monitoring occur in a common time period.

11. The method of claim 1, wherein the sonoporating comprises disrupting epithelial cell tight junctions within vascularized soft tissue.

12. The method of claim 2, wherein the administering the SIM product and the administering the dose of the drug occurs in a common injection to the patient.

13. A sonoporation system for sonoporation of a target treatment area to tissue permeability in the target treatment area, comprising:
a size-isolated microbubble (SIM) product comprising at least 80 volume percent microbubbles having a diameter of not less than about 4 μm and not more than about 5 μm at a concentration of not less than about 1.0×10⁹ microbubbles/mL; and
an ultrasound device operative to target the target treatment area with targeted ultrasound energy to cause sonoporation in the target treatment area in response to cavitation of the microbubbles of the SIM product.

14. The sonoporation system of claim 13, further comprising:
a dose of a therapeutic agent having a dose not greater than about 50% of an approved dose of the therapeutic agent.

15. The sonoporation system of claim 14, wherein the SIM product and the dose of the therapeutic agent are provided in a common administration vessel for simultaneous administration to a patient.

16. The sonoporation system of claim 14, wherein the therapeutic agent comprises a chemotherapy agent, and wherein the target treatment area comprises a vascularized soft tissue tumor.

17. The sonoporation system of claim 13, wherein the ultrasound device is operative to identify the target treatment area from ultrasound imaging generated by the ultrasound device.

18. The sonoporation system of claim 13, wherein the ultrasound device is operative to monitor the sonoporation of the target treatment area using ultrasound imagining generated by the ultrasound device.

19. The sonoporation system of claim 13, wherein the sonoporation in the target treatment area disrupts epithelial cell tight junctions within vascularized soft tissue.
